SEQUENCE LISTING (12 sequences)

SEQ ID NO: 1 - DNA, 18 bp, synthetic construct
  source 1..18: mol_type = other DNA
taggcaccagggtgtgat

SEQ ID NO: 2 - DNA, 16 bp, synthetic construct
  source 1..16: mol_type = other DNA
ctcctcaggggccaca

SEQ ID NO: 3 - DNA, 23 bp, synthetic construct
  source 1..23: mol_type = other DNA
gacgaagagcccttacagttcca

SEQ ID NO: 4 - DNA, 20 bp, synthetic construct
  source 1..20: mol_type = other DNA
tctgcagtgcctccactatg

SEQ ID NO: 5 - DNA, 19 bp, synthetic construct
  source 1..19: mol_type = other DNA
cctggtccctctggaaatg

SEQ ID NO: 6 - DNA, 20 bp, synthetic construct
  source 1..20: mol_type = other DNA
ggaagcctctttctcctctc

SEQ ID NO: 7 - DNA, 22 bp, synthetic construct
  source 1..22: mol_type = other DNA
caagcttccttgtgcaagtgtc

SEQ ID NO: 8 - DNA, 21 bp, synthetic construct
  source 1..21: mol_type = other DNA
ttcatcttttggggtccgtca

SEQ ID NO: 9 - DNA, 21 bp, synthetic construct
  source 1..21: mol_type = other DNA
ttcctctctgcaagagacttc

SEQ ID NO: 10 - DNA, 20 bp, synthetic construct
  source 1..20: mol_type = other DNA
gttgggagtggtatcctctg

SEQ ID NO: 11 - DNA, 22 bp, synthetic construct
  source 1..22: mol_type = other DNA
aacctggggaatgtctcaaagt

SEQ ID NO: 12 - DNA, 20 bp, synthetic construct
  source 1..20: mol_type = other DNA
ggtgatgcacacctctgtga

(19) United States
(12) Patent Application Publication
(10) Pub. No.: US 2025/0228801 A1
(43) Pub. Date: Jul. 17, 2025

(54) USE OF Galectin-1 INHIBITOR IN PREPARATION OF DRUG FOR TREATING PULMONARY FIBROSIS

(71) Applicant: Institute of Basic Medical Sciences, CAMS, Beijing (CN)

(72) Inventors: Jing Wang, Beijing (CN); Meiyue Song, Beijing (CN); Jiaxin Wang, Beijing (CN); Xue Wang, Beijing (CN)

(73) Assignee: Institute of Basic Medical Sciences, CAMS, Beijing (CN)

(21) Appl. No.: 18/741,100

(22) Filed: Jun. 12, 2024

(30) Foreign Application Priority Data
Jan. 12, 2024 (CN) 202410051956.8

(51) Int. Cl. A61K 31/165; A61K 38/46; A61K 38/48; A61P 11/00

(52) U.S. Cl. CPC A61K 31/165; A61K 38/465; A61K 38/48; A61P 11/00

(57) ABSTRACT

Provided is a use of a Galectin-1 inhibitor in preparation of a drug for treating pulmonary fibrosis. Taking silicosis model mice as a subject, administration of a Galectin-1 inhibitor OTX008 to silicosis mice can effectively alleviate the progression of pulmonary fibrosis. Studies have shown that the lung function of mice with silicosis is improved significantly; transcription levels of inflammatory factors Il-1β and Il-6 in the lung tissue of silicosis mice are decreased, concentrations of IL-1β and IL-6 in the alveolar lavage fluid are also decreased, and the infiltration of inflammatory cells is decreased. In the lung tissue of silicosis mice, the transcription levels of fibrosis factors Col-I and Fn-1 are decreased, the collagen-specific amino acid hydroxyproline is decreased, the fibrosis lesions are decreased, and the degree of lesions is alleviated. Therefore, the Galectin-1 inhibitor can be used as a new treatment strategy for pulmonary fibrosis.

### CROSS REFERENCE TO RELATED APPLICATION

This patent application claims the benefit and priority of Chinese Patent Application No. 2024100519568, filed with the China National Intellectual Property Administration on Jan. 12, 2024, the disclosure of which is incorporated by reference herein in its entirety as part of the present application.

### REFERENCE TO SEQUENCE LISTING

A computer readable XML file entitled “SEQUENCE LISTING”, that was created on Apr. 26, 2024, with a file size of about 11440 bytes, contains the sequence listing for this application, has been filed with this application, and is hereby incorporated by reference in its entirety.

### TECHNICAL FIELD

The present disclosure belongs to the technical field of biomedicine, and specifically relates to use of a Galectin-1 inhibitor in preparation of a drug for treating pulmonary fibrosis.

### BACKGROUND

Interstitial lung disease (ILD) is a general term for a group of diffuse pulmonary diseases involving the pulmonary interstitium, alveoli, and/or bronchioles. The ILD has many classifications and complex causes. The most representative ones are idiopathic pulmonary fibrosis (IPF) with unknown cause and pneumoconiosis with clear cause. Pneumoconiosis is one of the most important occupational diseases in the world. This disease is mainly caused by workers being exposed to a large amount of production dust for a long time during work, and the dust is deposited and retained in their lungs, eventually leading to diffuse fibrosis of the lung tissue. Pneumoconiosis is still a disease that has no medical end. In terms of non-drug treatment, whole lung alveolar lavage can only remove an extremely small amount of dust particles but cannot stop the progression of pneumoconiosis; while lung transplantation cannot be used as a routine treatment due to high surgical cost, lack of lung sources, and high risks. In terms of drug treatment, the efficacy and mechanism of a traditional drug tetrandrine is still unclear. Stem cell therapy has a certain potential in the treatment of pneumoconiosis, but its specific mechanism is not fully understood. Since the safety and effectiveness are still under study, the stem cell therapy currently cannot meet clinical demands. In addition, pneumoconiosis responds poorly to hormone treatment, which is closely related to the pathological characteristics of pneumoconiosis as shown by research reports. Faced with the current situation of no available treatment for pneumoconiosis, there is an urgent need to further explore the specific mechanisms of the occurrence and development of pneumoconiosis, so as to find potential new drug targets and promote the clinical treatment of pulmonary fibrosis.

Recent research evidence shows that Galectin-1 is expressed or overexpressed in tumors and/or surrounding tissues, and is considered a biomarker for the diagnosis, prognosis, and treatment of malignant tumors. The Galectin-1 can participate in the adhesion and migration of tumor cells, cell transformation, invasion of tumor cells into surrounding normal tissues, tumor blood vessel growth, and tumor immune evasion. In addition, the Galectin-1 can also regulate T cells, B cells, macrophages, granulocytes and other immune cells, promoting immune tolerance and down-regulating innate and adaptive immune responses. Galectin-1 plays an important role in various diseases such as autoimmune diseases, bacterially and virally infectious diseases, and neurological diseases. However, there is a lack of relevant research on the role of Galectin-1 in pulmonary fibrosis. Therefore, it is of great significance to explore whether targeting Galectin-1 can be used in the treatment of pulmonary fibrosis, thus urgently requiring further exploration.

### SUMMARY

In view of this, an objective of the present disclosure is to provide use of a Galectin-1 inhibitor in preparation of a drug for treating pulmonary fibrosis. A Galectin-1 inhibitor OTX008 can effectively improve pulmonary dysfunction, pulmonary inflammation, and pulmonary fibrosis.

To achieve the above objective, the present disclosure provides the following technical solutions:

The present disclosure provides use of a Galectin-1 inhibitor in preparation of a drug for treating pulmonary fibrosis.

Preferably, the Galectin-1 inhibitor is one or two selected from the group consisting of a modulator capable of reducing a Galectin-1 expression level and a modulator capable of reducing a Galectin-1 product.

More preferably, the modulator capable of reducing the Galectin-1 expression level includes OTX008.

More preferably, an OTX008 solution has a concentration of 0.5 mg/mL to 3 mg/mL.

More preferably, the OTX008 solution is prepared with a mixture including 5% to 15% of dimethyl sulfoxide (DMSO) and 80% to 95% of corn oil as a solvent.

More preferably, the Galectin-1 includes a protease and a nuclease that degrade the Galectin-1 product.

More preferably, the drug ameliorates pulmonary dysfunction.

More preferably, the drug ameliorates pulmonary inflammation and pulmonary fibrosis.

The present disclosure further provides a drug for treating pulmonary fibrosis, including an active ingredient and a pharmaceutically acceptable carrier, where the active ingredient includes the modulator OTX008 capable of reducing the Galectin-1 expression level.

Preferably, a dosage form of the drug is selected from the group consisting of a capsule, a powder, a tablet, and a solution.

Compared with the prior art, the present disclosure has the following beneficial effects:

The present disclosure provides use of a Galectin-1 inhibitor in preparation of a drug for treating pulmonary fibrosis. In the present disclosure, silicosis model mice are used as a subject to allow research. Administration of the Galectin-1 inhibitor OTX008 to silicosis mice can significantly inhibit the level of Galectin-1 and effectively alleviate the progression of silicosis. Specifically, when the Galectin-1 inhibitor OTX008 is administered, the Lgals1 transcription level in the lung tissue of silicosis mice is decreased, while the Galectin-1 translation level in the alveolar lavage fluid is also decreased. Meanwhile, the lung function of silicosis mice is significantly improved, including lung volume indicators such as inspiratory capacity (IC) and pulmonary ventilation function tests such as inspiratory resistance (RI), dynamic compliance (Cdyn), and quasi-static compliance (Cchord). The transcription levels of inflammatory factors Il-1β and Il-6 in the lung tissue of silicosis mice are decreased; the concentrations of inflammatory factors IL-1β and IL-6 in alveolar lavage fluid are also decreased, and inflammatory infiltration in the lungs is reduced; the transcription levels of fibrosis factors Fn-1 and Col-I are decreased, collagen-specific amino acid hydroxyproline (HYP) is decreased, fibrosis lesions are decreased, and the degree of lesions is alleviated. Therefore, the Galectin-1 inhibitor can be used as a new treatment strategy for pulmonary fibrosis.

### BRIEF DESCRIPTION OF THE DRAWINGS

FIG. 1 shows the transcript levels of Lgals1 in lung tissue of mice in different experimental groups, **P<0.01, ***P<0.001;

FIG. 2 shows the concentration of Galectin-1 in the bronchoalveolar lavage fluid of mice in different experimental groups, ***P<0.001;

FIGS. 3A-3D show influence of the Galectin-1 inhibitor OTX008 on lung function damage in silicosis mice, where FIG. 3A is mouse IC; FIG. 3B is RI; FIG. 3C is Cdyn; FIG. 3D is Cchord; *P<0.5, **P<0.01, ***P<0.001;

FIGS. 4A-4D show influence of the Galectin-1 inhibitor OTX008 on the levels of inflammatory factors in silicosis mice, where FIG. 4A is the transcription level of Il-1β in mouse lung tissue; FIG. 4B is the transcription level of Il-6 in mouse lung tissue; FIG. 4C is the concentration of IL-1β in mouse bronchoalveolar lavage fluid; FIG. 4D is the concentration of IL-6 in mouse bronchoalveolar lavage fluid; *P<0.5, **P<0.01, ***P<0.001;

FIGS. 5A-5D show the HE staining of the lung tissue of silicosis mice after administration of the Galectin-1 inhibitor OTX008;

FIGS. 6A-6C show influence of the Galectin-1 inhibitor OTX008 on pulmonary fibrosis in silicosis mice, where FIG. 6A is the transcription level of Col-I in mouse lung tissue; FIG. 6B is the transcription level of Fn-1 in mouse lung tissue; FIG. 6C is the content of HYP in mouse lung tissue; *P<0.5, **P<0.01, ***P<0.001; and

FIGS. 7A-7D show the Masson staining of the lung tissue of silicosis mice after administration of the Galectin-1 inhibitor OTX008.

### DETAILED DESCRIPTION OF THE EMBODIMENTS

The present disclosure provides use of a Galectin-1 inhibitor in preparation of a drug for treating pulmonary fibrosis. In the present disclosure, the pulmonary fibrosis mainly refers to fibrotic interstitial lung disease (f-ILD), a heterogeneous disease characterized by obvious fibrosis and inflammation in the lung interstitium. A type of the pulmonary fibrosis is not particularly limited. All types of f-ILD that can be diagnosed according to the “Expert Consensus on Diagnosis and Treatment of Interstitial Lung Disease” are within the scope of the present disclosure. Such types preferably include IPF, fibrotic non-specific interstitial pneumonia, chronic hypersensitivity pneumonitis, connective tissue disease-related interstitial lung disease, and pneumoconiosis.

In the present disclosure, the Galectin-1 inhibitor is one or two selected from the group consisting of a modulator capable of reducing a Galectin-1 expression level and a modulator capable of reducing a Galectin-1 product.

In the present disclosure, the modulator capable of reducing the Galectin-1 expression level includes OTX008. The OTX008 is a calixarene derivative, as well as a selective inhibitor of Galectin-1, and has anti-tumor activity. There is no special limitation on a source of the Galectin-1 inhibitor OTX008, which can be purchased through conventional commercial channels. The Galectin-1 inhibitor OTX008 is purchased from MedChemExpress (MCE).

In the present disclosure, the OTX008 solution has a concentration of preferably (0.5-3) mg/mL, more preferably (1-2) mg/mL, and even more preferably 1.5 mg/mL.

More preferably, the OTX008 solution is prepared with a mixture including preferably 5% to 15% of DMSO and 80% to 95% of corn oil, more preferably 10% of DMSO and 90% of corn oil as a solvent. For example, 1.5 mg of the OTX008 is dissolved in 100 μL of the DMSO, and then dissolved in 900 μL of the corn oil to bring a final concentration to 1.5 mg/mL.

In the present disclosure, the Galectin-1 inhibitor includes a protease and a nuclease that degrade the Galectin-1 product.

In the present disclosure, the drug ameliorates pulmonary dysfunction.

In the present disclosure, the drug ameliorates pulmonary inflammation and pulmonary fibrosis.

The present disclosure further provides a drug for treating pulmonary fibrosis, including an active ingredient and a pharmaceutically acceptable carrier, where the active ingredient includes the modulator OTX008 capable of reducing the Galectin-1 expression level. The carrier includes a buffer, a vehicle, a stabilizer, or a preservative, for example, starch, lactose, magnesium stearate, sodium sulfite, and ascorbic acid. Routes of administration of the medicament provided by the present disclosure may include oral, intravenous, parenteral, intramuscular, subcutaneous, intraperitoneal, intranasal, rectal, or topical administration. In the present disclosure, a dosage of the medicament provided by the present disclosure may be determined by disease type, disease severity, route of administration, age, gender and health conditions of patients.

In the present disclosure, a dosage form of the drug is selected from the group consisting of a capsule, a powder, a tablet, and a solution.

The technical solution provided by the present disclosure will be described in detail below with reference to the examples, but they should not be construed as limiting the claimed scope of the present disclosure.

### Example 1

Experimental Study on the Treatment of Silicosis with Galectin-1 Inhibitor OTX008

### (1) Construction of a Mouse Model of Silicosis and Administration of OTX008

Male C57BL/6J mice (aged 8 weeks and weighing 25-30 g) were selected and housed in an SPF grade laboratory animal room, and the model of silicosis was constructed by one-time intratracheal instillation of silica (Si); the mice were divided into four groups (n=9):
1) PBS+vehicle group: tracheal infusion of PBS, intraperitoneal injection of vehicle (10% DMSO+90% corn oil) 2 weeks later, once every other day for 3 weeks;
2) PBS+OTX008 group: PBS was given by tracheal infusion, and 2 weeks later, OTX008 (5 mg/kg) was given by intraperitoneal injection, once every other day for 3 weeks;
3) Si+vehicle group: Silica suspension (300 mg/mL, 40 μL) was given tracheal infusion, and vehicle (10% DMSO+90% corn oil) was given intraperitoneal injection 2 weeks later, once every other day for 3 weeks; and
4) Si+OTX008 group: Silica suspension (300 mg/mL, 40 μL) was given tracheal infusion, and OTX008 (5 mg/kg) was given intraperitoneal injection 2 weeks later, once every other day for 3 weeks.

All mice were sacrificed after the administration, and the corresponding samples were collected for detection.

### (2) Pulmonary Function Test

Anesthetized mice were fixed on an experimental table, and mouse lung function was detected by a pulmonary function testing system (DSI Buxco, USA). Before experiment, the mouse was anesthetized by intraperitoneal injection of 0.4 mL/100 g 2% pentobarbital, tracheotomy was performed, a trachea cannula was inserted, and a ventilator was connected. Next, FRC, PV, FV, and RC were automatically tested by a PET system. Indicators closely related to lung function changes in silicosis for statistical analysis, including lung volume indicators such as IC, and pulmonary ventilation function tests such as RI, Cdyn, and Cchord.

### (3) Pathological Staining

The left lung was fixed in 4% paraformaldehyde for 72 h, dehydrated, paraffin-embedded, sectioned (to 5 μm), and subjected to HE staining and Masson staining, respectively. The HE staining was conducted to calculate the inflammation score based on Szapiel's score. The scoring method included no inflammation (grade 0), mild (grade 1), moderate (grade 2), and severe (grade 3); Masson staining was conducted to evaluate the degree of fibrosis based on the King score. Specifically, different silicosis nodules were evaluated for fibrosis degree according to King's method, with levels ranging from 0 to 5. Each silicosis nodule was assigned a corresponding fibrosis damage score, which was calculated as the fibrosis level score (0-5) multiplied by its percentage of the total area of the tissue section. The sections were scanned, photographed and counted by a 3D HISTECH digital slide scanner.

### (4) ELISA

Concentrations of inflammatory factors IL-1β and IL-6 as well as Galectin-1 in mouse BALF were detected by using ELISA kits.

(5) qPCR

Lung tissue RNA of all mice was extracted; cDNA was obtained by using a reverse transcription kit (KR103, Tiangen Biotechnology, Beijing, China); qPCR was conducted by using a SYBR Green I Q-PCR Kit (TransGen Biotech, Beijing China); data collection and analysis were conducted by Bio-Rad IQ5 system.

TABLE 1
Primer sequences (5′ to 3′)

| | | | |
|---|---|---|---|
| β-actin | F | TAGGCACCAGGGTGTGAT | SEQ ID NO: 1 |
| | R | CTCCTCAGGGGCCACA | SEQ ID NO: 2 |
| Fn-1 | F | GACGAAGAGCCCTTACAGTTCCA | SEQ ID NO: 3 |
| | R | TCTGCAGTGCCTCCACTATG | SEQ ID NO: 4 |
| Col-I | F | CCTGGTCCCTCTGGAAATG | SEQ ID NO: 5 |
| | R | GGAAGCCTCTTTCTCCTCTC | SEQ ID NO: 6 |
| Il-1β | F | CAAGCTTCCTTGTGCAAGTGTC | SEQ ID NO: 7 |
| | R | TTCATCTTTTGGGGTCCGTCA | SEQ ID NO: 8 |
| Il-6 | F | TTCCTCTCTGCAAGAGACTTC | SEQ ID NO: 9 |
| | R | GTTGGGAGTGGTATCCTCTG | SEQ ID NO: 10 |
| Lgals1 | F | AACCTGGGGAATGTCTCAAAGT | SEQ ID NO: 11 |
| | R | GGTGATGCACACCTCTGTGA | SEQ ID NO: 12 |

### (6) Detection of HYP Content

30 mg of mouse lung tissue was detected using an HYP kit (NBP2-59747, Novus Biologicals, Littleton, CO, USA).

### (7) Analysis of Results

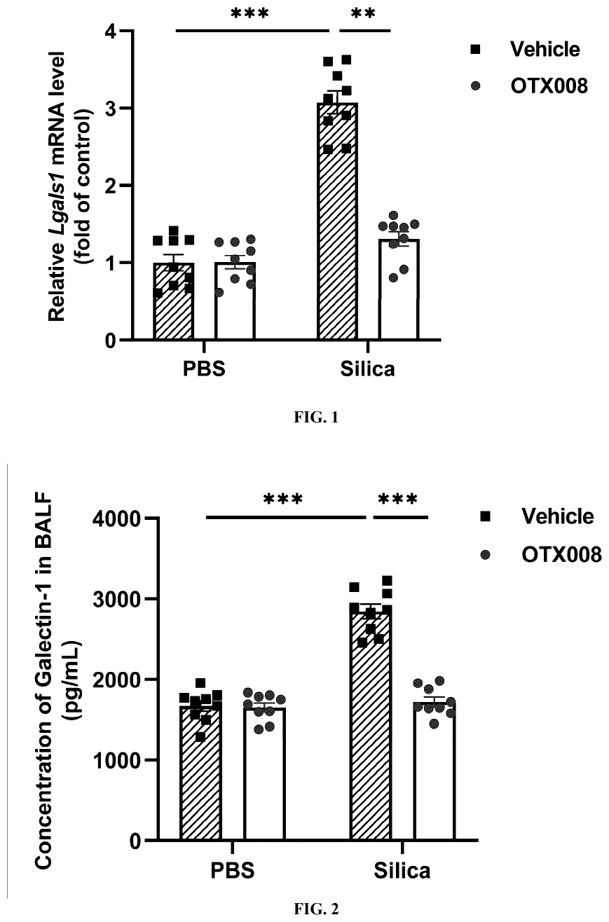

As shown in FIG. 1 to FIG. 2, the transcription and translation levels of Galectin-1 were significantly up-regulated in the silicosis mouse model, indicating that Galectin-1 might be involved in the occurrence and development of silicosis. When the Galectin-1 inhibitor OTX008 was administered to silicosis mice, it effectively inhibited the levels of Galectin-1.

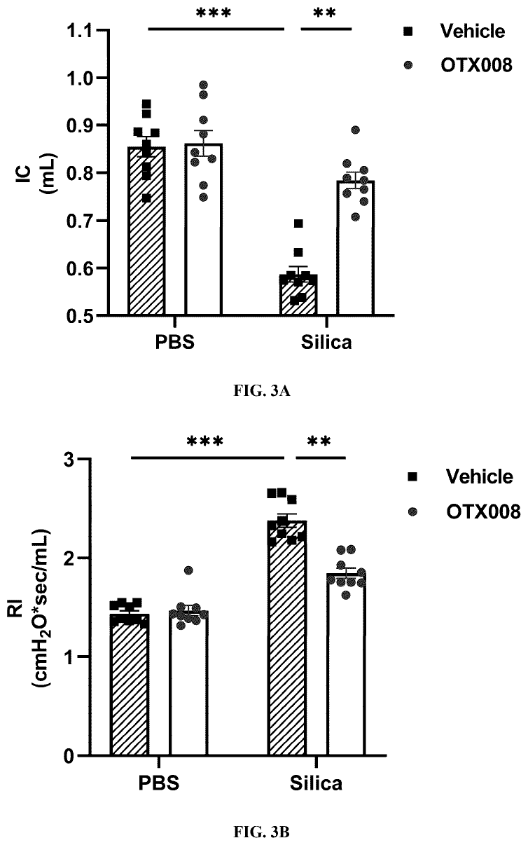

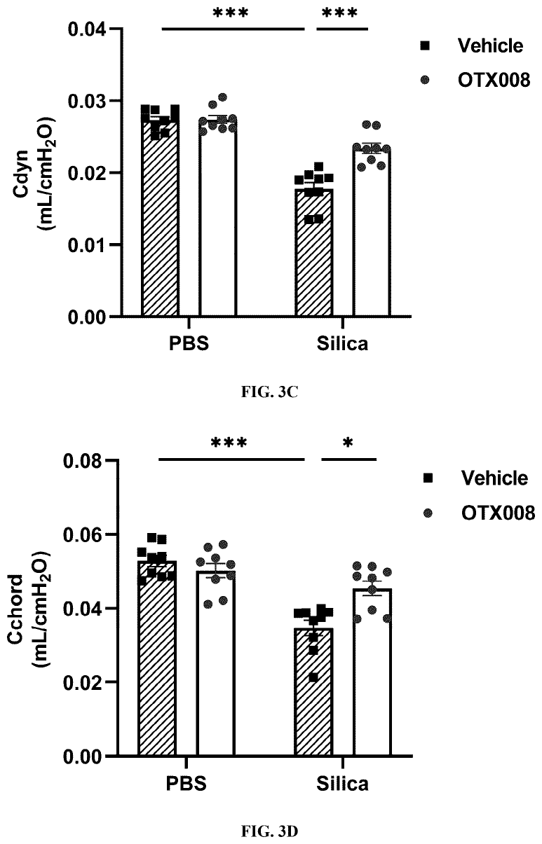

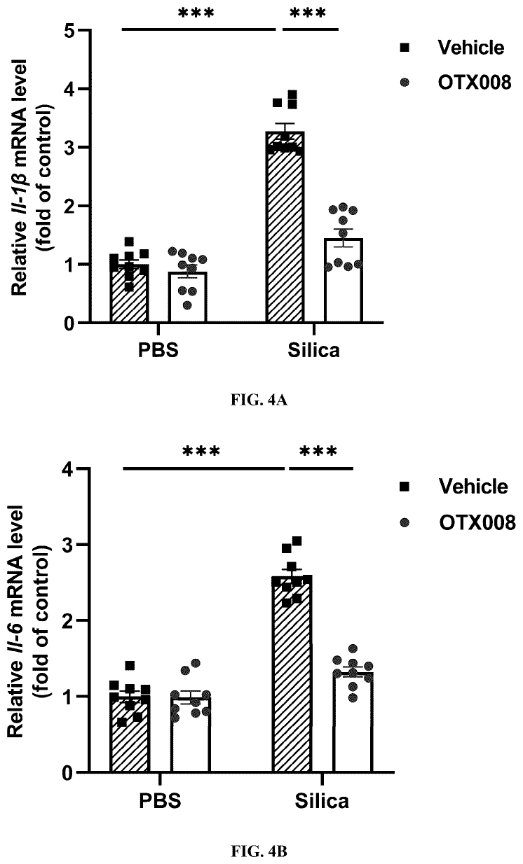

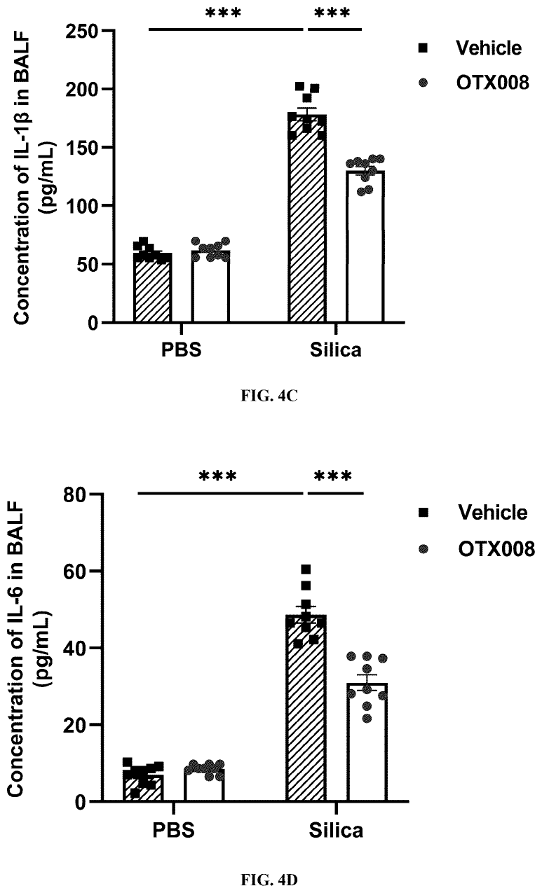

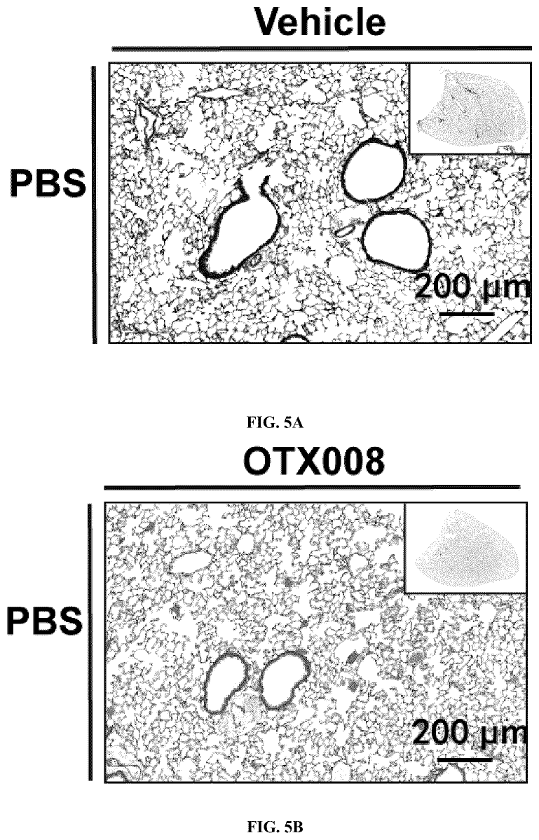

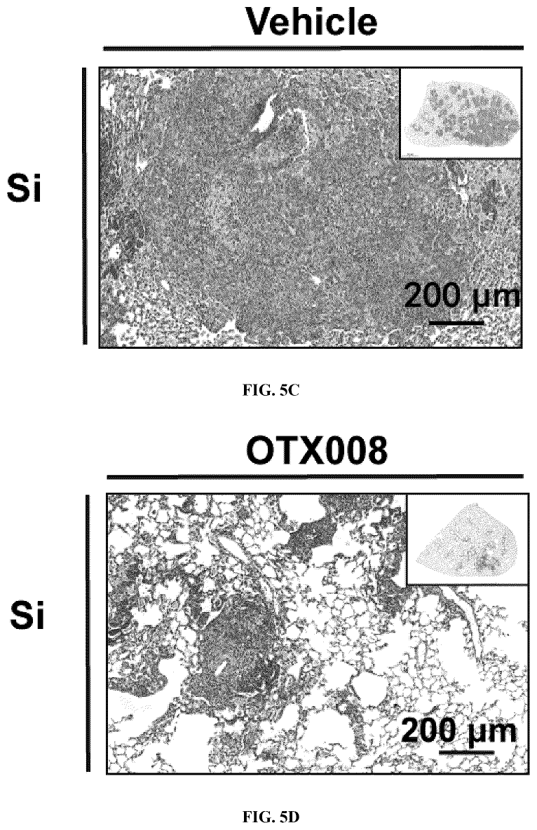

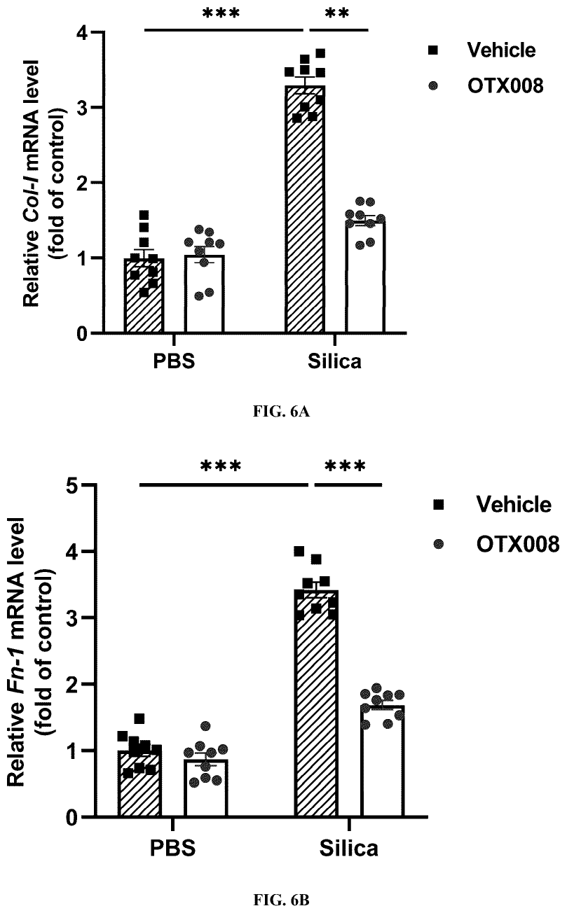

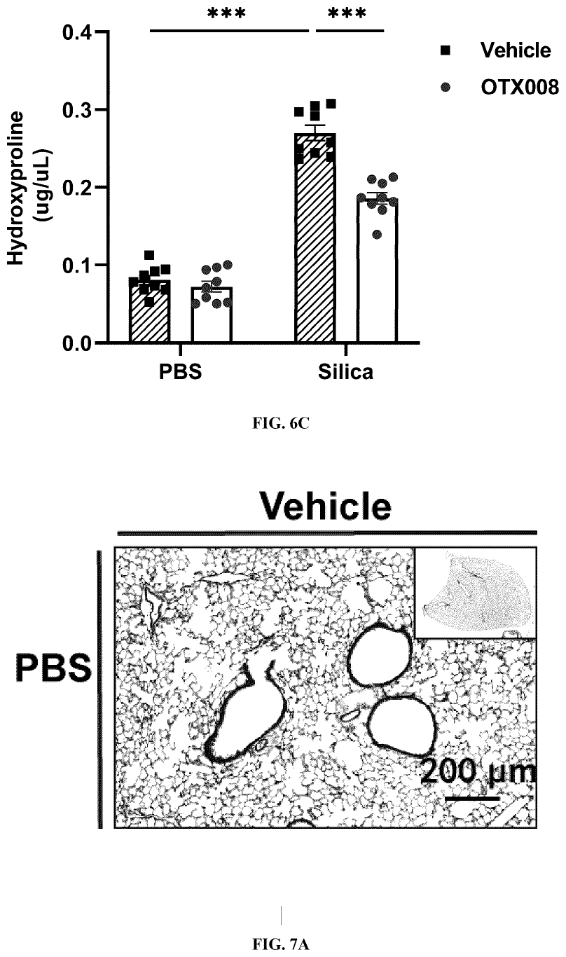

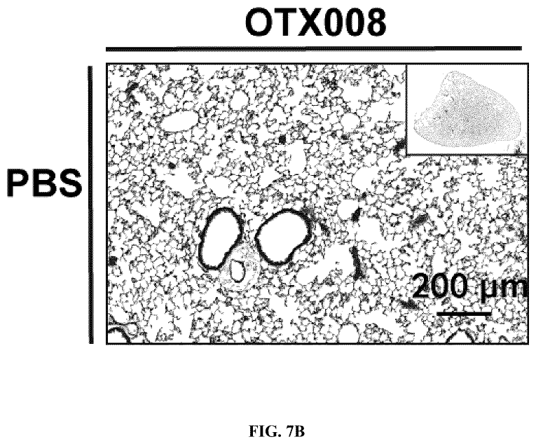

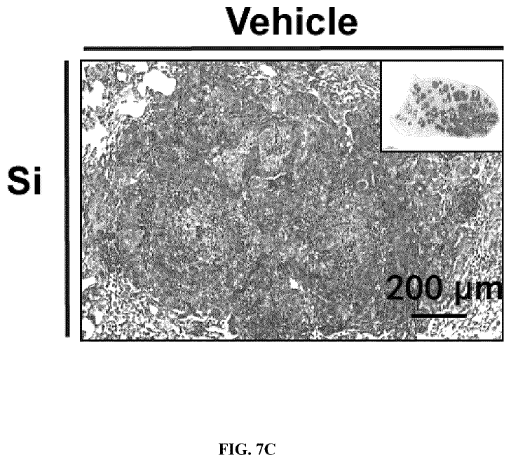

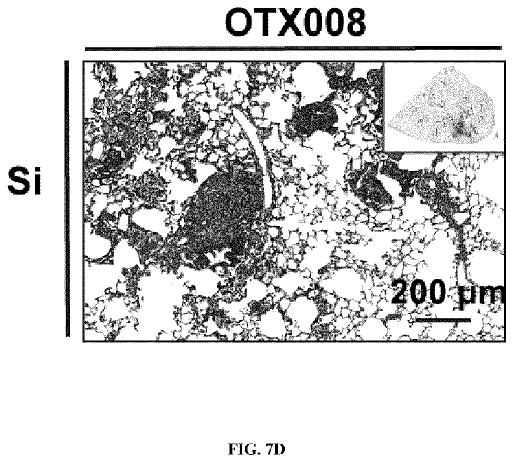

In addition, administration of OTX008 to silicosis mice could effectively alleviate the progression of pulmonary fibrosis. The results were shown in FIGS. 3A-3D to FIGS. 7A-7D: after administration of the Galectin-1 inhibitor OTX008, the lung function of IPF mice improved significantly, including lung volume indicators such as IC (FIG. 3A), and lung ventilation function tests such as RI (FIG. 3B), Cdyn (FIG. 3C) and Cchord (FIG. 3D); the transcription levels of inflammatory factors Il-1β (FIG. 4A), Il-6 (FIG. 4B) were decreased in IPF mice; the concentrations of inflammatory factors IL-1β (FIG. 4C) and IL-6 (FIG. 4D) in the alveolar lavage fluid decreased (FIGS. 5A-5D). In the lung tissue of silicosis mice, the transcription levels of fibrosis factors Col-I (FIG. 6A) and Fn-1 (FIG. 6B) were decreased, the collagen-specific amino acid HYP (FIG. 6C) was decreased, the Masson staining showed that the fibrosis lesions were reduced (FIGS. 7A-7D) and the degree of the disease was alleviated. Therefore, the Galectin-1 inhibitor can be used as a new treatment strategy for pulmonary fibrosis.

The above descriptions are merely preferred implementations of the present disclosure. It should be noted that a person of ordinary skill in the art may further make several improvements and modifications without departing from the principle of the present disclosure, but such improvements and modifications should be deemed as falling within the protection scope of the present disclosure.

What is claimed is:

1. A method for preparation of a drug for treating pulmonary fibrosis using a Galectin-1 inhibitor.

2. The method according to claim 1, wherein the Galectin-1 inhibitor is one or two selected from the group consisting of a modulator capable of reducing a Galectin-1 expression level and a modulator capable of reducing a Galectin-1 product.

3. The method according to claim 2, wherein the modulator capable of reducing the Galectin-1 expression level comprises OTX008.

4. The method according to claim 3, wherein an OTX008 solution has a concentration of 0.5 mg/mL to 3 mg/mL.

5. The method according to claim 4, wherein the OTX008 solution is prepared with a mixture comprising 5% to 15% of dimethyl sulfoxide (DMSO) and 80% to 95% of corn oil as a solvent.

6. The method according to claim 2, wherein the Galectin-1 comprises a protease and a nuclease that degrade the Galectin-1 product.

7. The method according to claim 1, wherein the drug ameliorates pulmonary dysfunction.

8. The method according to claim 1, wherein the drug ameliorates pulmonary inflammation and pulmonary fibrosis.

9. A drug for treating pulmonary fibrosis, comprising an active ingredient and a pharmaceutically acceptable carrier, wherein the active ingredient comprises the modulator OTX008 capable of reducing the Galectin-1 expression level according to claim 3.

10. The drug according to claim 9, wherein a dosage form of the drug is selected from the group consisting of a capsule, a powder, a tablet, and a solution.

11. The method according to claim 2, wherein the drug ameliorates pulmonary dysfunction.

12. The method according to claim 3, wherein the drug ameliorates pulmonary dysfunction.

13. The method according to claim 4, wherein the drug ameliorates pulmonary dysfunction.

14. The method according to claim 5, wherein the drug ameliorates pulmonary dysfunction.

15. The method according to claim 6, wherein the drug ameliorates pulmonary dysfunction.

16. The method according to claim 2, wherein the drug ameliorates pulmonary inflammation and pulmonary fibrosis.

17. The method according to claim 3, wherein the drug ameliorates pulmonary inflammation and pulmonary fibrosis.

18. The method according to claim 4, wherein the drug ameliorates pulmonary inflammation and pulmonary fibrosis.

19. The method according to claim 5, wherein the drug ameliorates pulmonary inflammation and pulmonary fibrosis.

20. The method according to claim 6, wherein the drug ameliorates pulmonary inflammation and pulmonary fibrosis.
